(19) United States
(12) Patent Application Publication
(10) Pub. No.: US 2013/0053704 A1
(43) Pub. Date: Feb. 28, 2013

(54) MEDICAL DEVICE FOR USE IN ASSESSING THE STATE OF A PERSON'S ENDOTHELIUM

(71) Applicants: Alan Bernjak, Lancaster (GB); Aneta Stefanovska, Lancaster (GB)

(72) Inventors: Alan Bernjak, Lancaster (GB); Aneta Stefanovska, Lancaster (GB)

(21) Appl. No.: 13/582,161

(22) PCT Filed: Feb. 16, 2011
(86) PCT No.: PCT/GB2011/050296

§ 371 Date: Nov. 6, 2012

(30) Foreign Application Priority Data
Mar. 2, 2010 (GB) 1003412.2

(51) Int. Cl. A61B 5/026; A61B 5/0265

(52) U.S. Cl. 600/479; 600/500

(57) ABSTRACT

Disclosed is a device for use in assessing the state of a person's endothelium, comprising first and second probes for attachment to the person's body, said first probe (1) comprising a portion for direct contact with the person's skin (3) to apply localised thermal stress to the skin, and a device for measuring microvascular perfusion (7), and said second probe being used to provide a timing reference signal. Also disclosed is a method of use.

Measurement of the propagation times of cardiac induced pulse waves along the arterial tree provides an important tool for studying arteries and the health thereof. It enables their viscoelastic properties to be quantified in terms of arterial stiffness or its inverse, arterial compliance. Arterial stiffness is increasingly used in clinical assessment and diagnosis on account of its role in the development of cardio-vascular disease.

Pulse transit time (PTT) is defined as the time taken for the pulse wave form generated by the heart to traverse a certain section of artery. It represents a simple, reproducible and non-invasive measure. Knowing the propagation distance, one can use the PTT to calculate the pulse wave velocity, which is generally accepted as being the most robust of the available indices of arterial stiffness.

Recent studies have shown a correlation between arterial stiffness and the endothelial function. It has been shown that changes in PTT can be used to measure the smooth muscle relaxation that occurs when a functioning endothelium is stimulated. Studies on children have related arterial stiffness directly to impaired endothelium function, for example, in cases of low birth weight or severe obesity. Several lines of evidence now support the hypothesis that endothelium regulates arterial stiffness by release of vasoactive mediators. Inhibition of basal nitric oxide (NO) production in the endothelium with L-NMMA, for example, increases the stiffness of the brachial artery. Acetylcholine (ACh), an endothelium dependent vasodilator, reduces stiffness in large arteries, but the reduction is inhibited by L-NMMA, an inhibitor of NO synthase.

Several techniques are known to measure pulse wave velocity. They evaluate PTT based on detecting blood pressure pulses using applanation tonometry, blood velocity pulses using Doppler ultrasound or volume changes by applying photoplethysmography.

Most clinically relevant techniques in the field are limited to the detection of stiffness in large arteries. It has recently been shown using photoplethysmography, that PTT to the small peripheral arteries can be used to assess stiffness of the lower body arteries. These transmit times include the time for the pulse to travel through the aorta, large peripheral arteries and small arteries on the tip of the finger or toe. However, the propagation of pulse waves through the network of capillaries has not been fully investigated in the prior art. In the micro-circulation, the pulsation of the heart is reduced compared to that in the arteries, so that the detection of pulse wave forms is not straightforward. One suitable method for this purpose is laser Doppler flowmetry (LDF) which enables non-invasive and continuous measurements of microvascular perfusion. This technique is widely used in the assessment of the dynamical properties of skin microcirculation, both in health and in various cardiovascular diseases.

None of the available prior art techniques permits a reliable and automated measure of the function and/or condition of the endothelium to be performed. The existing automated techniques for evaluation of endothelial function measure changes, such as volumetric or temperature changes in the peripheral arteries in response to occlusion of the brachial artery. In this way they introduce a disruption in the blood circulation and estimate the function of the endothelium based on an integrated response in the peripheral arteries. Embodiments of the present invention aim to address these and other shortcomings in the prior art, whether mentioned herein or not.

According to the present invention there is provided an apparatus and method as set forth in the appended claims. Other features of the invention will be apparent from the dependent claims, and the description which follows.

For a better understanding of the invention, and to show how embodiments of the same may be carried into effect, reference will now be made, by way of example, to the accompanying diagrammatic drawings in which:

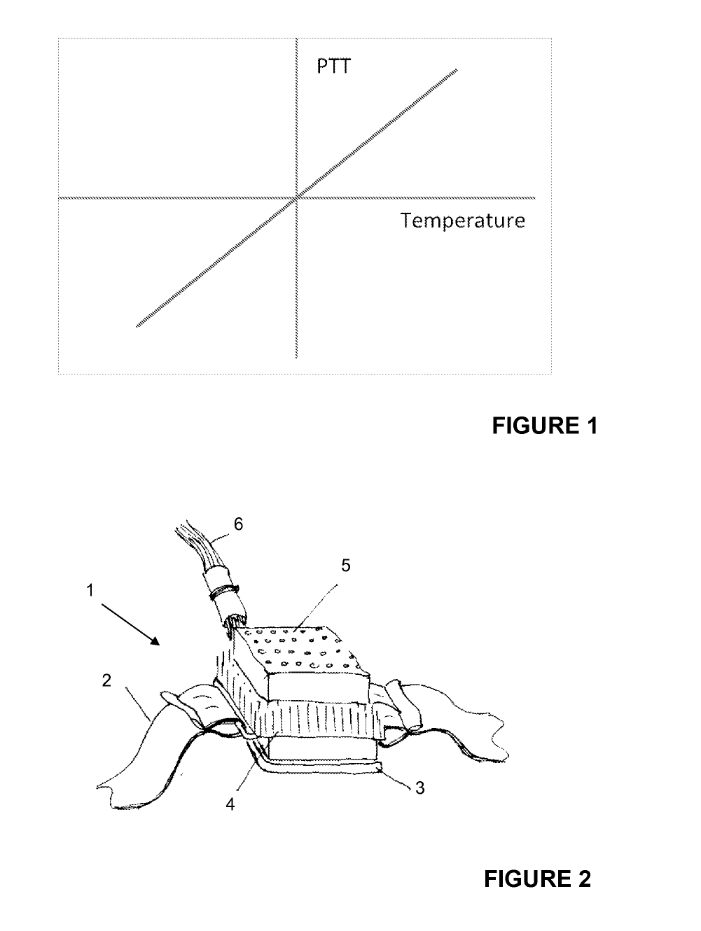

FIG. 1 shows an idealised plot of PTT versus temperature around basal skin temperature;

FIG. 2 shows a perspective view of a probe according to an embodiment of the invention;

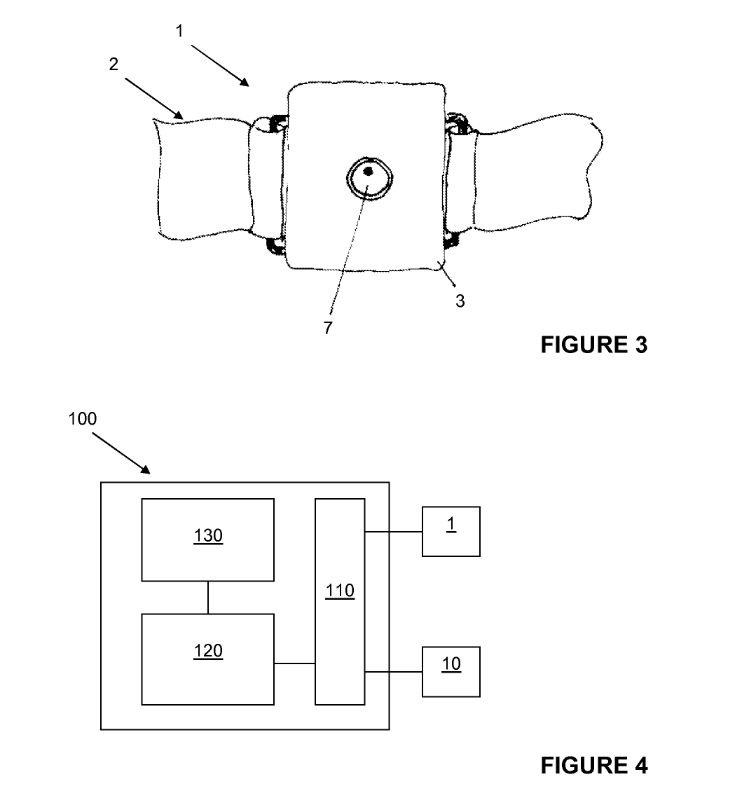

FIG. 3 shows a lower plan view of the probe of FIG. 2; and

FIG. 4 shows a system according to an embodiment of the present invention.

Embodiments of the present invention present a new device herein called an endotheliometer, which is a non-invasive instrument to measure local endothelium responsiveness. It is found empirically that endothelial malfunction is a component of vascular diseases such as heart attacks. The disclosed technique can easily be applied on parts of the surface of the body and in particular on all four extremities. Hence the functioning of the endothelium can be assessed in different parts of the body. Therefore measurements of endothelial reactivity of easily accessible sites can act as a proxy for the entire endothelium including critical organs.

Embodiments of the present invention are operable to measure the pulse transit time (PTT) from the skin microcirculation. More accurately, they are able to measure the variation of PTT during the relative heating or cooling of a local area of skin. A particular embodiment of the invention utilises a probe arranged in contact with the subject's skin to do this.

FIG. 1 shows an idealised graph representing the variation of PTT against temperature. The temperature ranges between 30° C. and 40° C. The graph shows that PTT increases with increasing temperature of the skin. For skin temperatures below 30° C. and above 40° C. the PTT becomes gradually saturated and a horizontal plateau is expected. In subjects with endothelial dysfunction however, a smaller and slower change in PTT is expected with temperature. Consequentially, the slope of the curve shown in FIG. 1 is smaller with endothelial dysfunction. Furthermore, the linear PTT-temperature relationship occurs within a narrower temperature range. By measuring these variations from the ideal response, it is possible to calculate various metrics which are indicative of endothelium responsiveness and, by extension, the state of the individual's entire vascular system.

In order to measure the PTT in a given patient or subject, an endotheliometer, according to an embodiment of the present invention, may be used. The endotheliometer comprises several different components. These are a control system which includes signal acquisition circuitry, signal processing circuitry and a means of reporting the result of the analysis to the attending physician or technician. The system also comprises a first probe for attachment to a patient, the first probe being connected electrically and/or optically to the control system. A second probe is also provided for use in providing a timing reference. The second probe may be one or more of suitable ECG electrodes and/or a blood pulse measurement device attached away from the measurement site.

Unlike some of the prior art systems available for use in detecting endothelial function there is no need for interruption of the blood circulation by using a cuff.

The first probe for attachment to the patient is shown in perspective view in FIG. 2 and in lower plan view in FIG. 3. From FIG. 2 it can be seen that the probe 1 comprises one or more straps 2 which enable the probe to be attached to the patient's arm or other suitable location. The lower surface of the probe 3 comprises a substantially planar copper plate for direct contact with the patient's skin. Situated above the copper plate are a plurality of cooling ribs, fins or other heat sink structures 4. Directly above the cooling ribs 4 is a cooling fan 5. The entire probe arrangement 1 is connected to the control system by one or more cables and/or optical fibres 6.

The lower view of the probe 1 in FIG. 3 clearly shows the extent of the copper plate 3 and the centrally located LDF probe 7.

In use, the probe 1 is attached to the patient at a suitable location, e.g. the arm, and local temperature stress is applied to the skin, and measurements of the changes in arrival time of pulse waves to the skin microcirculation at the location of temperature stress are made. In response to the localised temperature changes to the skin, the stiffness of the local microvasculature is affected, resulting in changed transition times of the propagating pulse waves. With decreased temperature, tonus of the smooth muscles surrounding the vessels increases and the velocity of the propagating pulse waves increases. With higher velocity, the transit time of the pulse shortens. Equally, when the skin is heated the tonus of the muscles decreases, the velocity of the propagating pulse waves decreases and their transit times lengthen accordingly.

In this way, by local stimulation of the skin microvasculature and detection of changes in the pulse transit time at the capillary level, said capillaries mostly consisting of endothelial structures, the responsiveness of the endothelium can be assessed.

In order to apply the localised temperature stress to the patient's skin, the probe 1 incorporates a copper plate 3 for direct contact with the patient's skin. Copper is chosen as it is a good conductor of heat, although other suitable materials, especially metals, could be used in its place. The copper plate 3 has an area of approximately 20 cm² and is able to control the temperature applied to the patient's skin in a range of approximately 20 to 45° C. Positioned at the centre, or near the centre of the copper plate 3 is the blood flow or LDF probe 7. This device is able to monitor perfusion in the temperature perturbed skin microcirculation. The temperature of the copper plate 3 is controlled using a thermoelectric temperature controller. The cooling ribs or fins 4 positioned above the copper plate allow heat to be radiated away from the patient's skin and this process is assisted by the cooling fan 5. Other cooling mechanisms may be utilised, including, for instance, Peltier effect devices.

The second probe used to provide a timing reference is also attached to the patient, some way from the positioning of the first probe. If the second probe comprises EGC electrodes, then standard adhesive electrodes may be used and may be attached to the body to measure the ECG signal and a distinctive R-peak which can be accurately localised.

The blood flow in the microcirculation is measured in the skin using the LDF device and preferably set with a low time constant of at most 0.03 seconds. LDF devices measure instantaneous blood flow based on the difference of frequency spectra of incident and back-scattered light from the tissue. An integration of the collected data is performed with a frequency given by the time constant. A lower time constant means more often integration and a more accurate detection of the instantaneous blood flow values. To precisely follow the waveforms of the cardiac-related pulses and to localise their characteristic features it is therefore preferable to use a LDF device with a time constant of at most 0.03 s.

A coherent laser light is delivered to the probe through an optical fibre and is then collected after scattering in the microvasculature. In this way, the average blood flow in the skin microvasculature (comprising arterioles, venules and capillary bed) is measured from a volume of about 1 mm³.

To complete the probe set-up, a temperature probe (not shown) is attached to the skin in the vicinity of the first probe 1 to monitor the skin temperature.

The coherent laser light is generated in the control system 100 shown in FIG. 4. The laser light is transferred to the LDF probe by means of a fibre optic cable comprised in the cable loom 6.

The control system 100 is also operable to process all of the measured data from the first and second probes and to provide some form of read out either directly to the physician or to a suitably programmed computing device for further analysis. The control system 100 comprises a signal conditioning unit 110 which is operable to pre-process the received signals from the probe, said pre-processing including amplification of the analogue data from the ECG electrodes of the second probe 10 and the calculation of the blood flow data from the signal received via the optical cable. All of the received signals, including the blood flow, temperature and ECG signals are digitised using an at least 16-bit ND conversion rate.

The digitised signals are processed in a signal processing unit 120, operable to process the digitised signals as will be described shortly.

In order to make measurements of a particular individual, the individual is placed in a supine position and allowed to relax for a few minutes prior to the measurement being taken. Once the various electrodes and probes have been attached to the individual, simultaneous and continual readings are taken of ECG, blood flow and skin temperature and recorded in the control system 100. The preferred protocol includes taking measurements of the basal condition i.e. without temperature variations being applied and also in response to temperatures changes up and down from this basal condition. Once the basal condition has been recorded, the temperature controller is operable to decrease the skin temperature by a few degrees, followed by a gradual increase in temperature to a few degrees above the basal level.

All the time that these conditions are changing, the control system 100 is operable to record the gathered data and to process it. It is found that the blood flow signal includes an oscillation which is found to be related to cardiac activity. There are also further variations of lower frequencies of oscillations and modulations resulting from physiological processes involved in the regulation of blood flow in the microcirculation. Their contribution to the blood flow in terms of power is lower compared to the component related to the cardiac activity and can be effectively removed by performing digital filtering using a bandpass filter. In this way the desired data relating to the cardiac activity can be extracted from the flow signal while the other frequency components are suppressed. A 4^{th} order Butterworth bandpass filter is used for filtering. Two cut-off frequencies are set which determine the frequency band that the filter passes through—the band that includes the cardiac component, while the frequencies above and below the band are eliminated. Because the cardiac frequency varies between different subjects, the cut-off frequencies are set at 0.2 Hz below and above the mean heart rate of the subject, which can be extracted from the ECG signal. After the filtering is performed, the maxima of the obtained pulsations are detected. These are then used as markers for the location of the pressure waves within the flow signal.

When the skin is heated, the amplitude of the blood flow and the amplitude of its fluctuations significantly increase and the shape of the pulse waveforms changes. Therefore, in the heating phase, the filtering applied is altered to allow for this. Before applying bandpass filtering as described above, the trend of the blood flow (that is the slow variation of the blood flow in response to the temperature change) is removed. The trend of the signal is first calculated by applying a moving average method with a 10 s rectangular window, and the trend is then subtracted from the original signal. In this way, the big amplitude variations of the flow signal are removed and the mean value of the resulting signal is zero.

PTT is defined as the delay between the R peak in the measured ECG and the location of the corresponding pulse wave in the blood flow signal. It is calculated in the signal processing unit 120 as the time elapsed between the position of the R peak and the location of the determined maximum of the pulse wave. By calculating the PTT between each R peak and its corresponding pulse wave in the whole set of recorded data, the evolution of PTT in time (and hence in this case, temperature), can be observed.

With the application of temperature stress, the propagation of the pulse waves through the microvasculature is affected. PTT decreases with cooling and increases with heating, compared to the basal condition. The responsiveness of the PTT to the variation of skin temperature is a measure of the endothelial reactivity, and three measures or metrics have been defined to relate this information.

The absolute PTT change is the difference between the maximal value of PTT during heating of the skin and the minimal value of PTT during cooling:

ΔPTT=max(PTTₕₑₐₜ)−min(PTT_{cool}) (1)

The normalised PTT change is the absolute PTT change normalised to the minimal value of PTT, that is the minimal PTT reached during cooling:

ΔPTTₙ=(max(PTTₕₑₐₜ)−min(PTT_{cool}))/min(PTT_{cool}) (2)

The highest rate of PTT change is the biggest change in PTT during the temperature increase. This point is detected as the maximal value of the PTT time series derivative during the transition from minimal to maximal skin temperature:

ΔPTTᵣ=dPTT(t)/dt (3)

The values of measures introduced above are directly related to the measurement setup and the state of the endothelium and its functioning. The absolute and normalised change in PTT depends on the range of temperature introduced to the skin while the rate of PTT change depends on the rate of temperature change generated by the controller. At a temperature range of the copper plate from 29° C. to 43° C. and the rate of temperature change about 0.45° C./s, the following values are expected for a normally functioning endothelium: absolute change of PTT from 0.08 s to 0.17 s, relative change of PTT from 0.21 to 0.4 and the rate of PTT change from 0.001 to 0.002. Impaired endothelium is less responsive to temperature changes and its response is slower compared to intact healthy endothelium. Therefore, the measures obtained in impaired endothelium are below the above ranges.

Among the presented measures, the rate of PTT change is expected to be the most indicative of endothelial function because it directly estimates not only how strong the endothelial reaction is in response to temperature change but also how fast the response is.

Attention is directed to all papers and documents which are filed concurrently with or previous to this specification in connection with this application and which are open to public inspection with this specification, and the contents of all such papers and documents are incorporated herein by reference.

All of the features disclosed in this specification (including any accompanying claims, abstract and drawings), and/or all of the steps of any method or process so disclosed, may be combined in any combination, except combinations where at least some of such features and/or steps are mutually exclusive.

Each feature disclosed in this specification (including any accompanying claims, abstract and drawings) may be replaced by alternative features serving the same, equivalent or similar purpose, unless expressly stated otherwise. Thus, unless expressly stated otherwise, each feature disclosed is one example only of a generic series of equivalent or similar features.

The invention is not restricted to the details of the foregoing embodiment(s). The invention extends to any novel one, or any novel combination, of the features disclosed in this specification (including any accompanying claims, abstract and drawings), or to any novel one, or any novel combination, of the steps of any method or process so disclosed.

1. A device for use in assessing the state of a person's endothelium, comprising first and second probes for attachment to the person's body, said first probe comprising a portion for direct contact with the person's skin to apply localised thermal stress to the skin, and a device for measuring microvascular perfusion, and said second probe being used to provide a timing reference signal.

2. The device of claim 1 wherein the second probe is attached to the person's body, remote from the first probe.

3. The device of claim 1 wherein the second probe is one of one or more ECG electrodes and a blood pulse measurement device.

4. The device of claim 1 wherein the device for measuring microvascular perfusion is a Laser Doppler Flowmetry (LDF) device.

5. The device of claim 4 wherein the LDF device is located substantially centrally within the portion for direct contact with the skin.

6. The device of claim 1 wherein the portion for direct contact with the skin comprises a substantially planar metallic plate.

7. The device of claim 6 wherein the metallic plate is provided with one or more cooling fins.

8. The device of claim 1 wherein the portion for direct contact with the skin is arranged to vary in temperature substantially in the range 20-45° C.

9. The device of claim 1 further comprising signal processing apparatus arranged to condition and digitise signals received from the first and second probes.

10. The device of claim 1, further comprising apparatus arranged to calculate at least one metric on the basis of signals measured from the first and second probes.

11. A method of assessing the state of a person's endothelium, comprising the use of a device as claimed in 1, comprising the steps of:
attaching the first probe to the person;
attaching the second probe to the person, at a location remote from the first probe;
taking basal readings from the first and second probes;
reducing the temperature applied to the person's skin, and taking further readings from the first and second probes; and
increasing the temperature and taking readings from the first and second probes at a plurality of different temperatures.
